SEQUENCE LISTING (11 sequences)

SEQ ID NO: 1 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 2 - protein, 422 aa, Vesicular stomatitis virus
MSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDK

SEQ ID NO: 3 - protein, 265 aa, Vesicular stomatitis virus
MDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 4 - protein, 2109 aa, Vesicular stomatitis virus
MEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNPPSDGIAQNVGIAITGISFWLSLMEKDIPLYQQCLAVIQQSFPIRWEAVSVKGGYKQKWSTRGDGLPKDTRISDSLAPIGNWIRSLELVRNQVRLNPFNEILFNQLCRTVDNHLKWSNLRRNTGMIEWINRRISKEDRSILMLKSDLHEENSWRD

SEQ ID NO: 5 - protein, 229 aa, Vesicular stomatitis virus
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 6 - protein, 498 aa, Lymphocytic choriomeningitis mammarenavirus
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 7 - protein, 498 aa, unidentified
  source 1..498: note = Dandenong virus
MGQLITMFEALPHIIDEVINIVIIVLVIITSIKAVYNFATCGIIALISFCLLAGRSCGLYGVTGPDIYKGLYQFKSVEFNMSQLNLTMPNACSANNSHHYISMGKSGLELTFTNDSIISHNFCNLTDGFKKKTFDHTLMSIVASLHLSIRGNTNYKAVSCDFNNGITIQYNLSFSDAQSAINQCRTFRGRVLDMFRTAFGGKYMRSGYGWKGSDGKTTWCSQTSYQYLIIQNRTWENHCEYAGPFGLSRVLFAQEKTKFLTRRLAGTFTWTLSDSSGTENPGGYCLTKWMLIAAELKCFGNTAVAKCNINHDEEFCDMLRLIDYNKAALKKFKEDVESALHLFKTTVNSLISDQLLMRNHLRDLMGVPYCNYSKFWYLEHVKTGDTSVPKCWLVSNGSYLNETHFSDQIEQEADNMITEMLRKDYIKRQGSTPLALMDLLMFSTSAYLISVFLHLMKIPTHRHIKGGTCPKPHRLTSKGICSCGAFKVPGVKTVWKRR

SEQ ID NO: 8 - protein, 489 aa, Mopeia mammarenavirus
MGQIVTFFQEVPHILEEVMNIVLMTLSILAILKGIYNVMTCGIIGLITFLFLCGRSCSSIYKDNYEFFSLDLDMSSLNATMPLSCSKNNSHHYIQVGNETGLELTLTNTSIIDHKFCNLSDAHRRNLYDKALMSILTTFHLSIPDFNQYEAMSCDFNGGKISIQYNLSHSNYVDAGNHCGTIANGIMDVFRRMYWSTSLSVASDISGTQCIQTDYKYLIIQNTSWEDHCMFSRPSPMGFLSLLSQRTRNFYISRRLLGLFTWTLSDSEGNDMPGGYCLTRSMLIGLDLKCFGNTAIAKCNQAHDEEFCDMLRLFDFNKQAISKLRSEVQQSINLINKAVNALINDQLVMRNHLRDLMGIPYCNYSKFWYLNDTRTGRTSLPKCWLVTNGSYLNETQFSTEIEQEANNMFTDMLRKEYEKRQSTTPLGLVDLFVFSTSFYLISVFLHLIKIPTHRHIKGKPCPKPHRLNHMAICSCGFYKQPGLPTQWKR

SEQ ID NO: 9 - DNA, 24 bp, synthetic construct
  source 1..24: mol_type = other DNA
agtaccggaggattgacgactaat

SEQ ID NO: 10 - DNA, 20 bp, synthetic construct
  source 1..20: mol_type = other DNA
tcaaaccatccgagccattc

SEQ ID NO: 11 - DNA, 24 bp, synthetic construct
  source 1..24: mol_type = other DNA
accgccacaaggcagagatgtggt

(19) United States
(12) Patent Application Publication
(10) Pub. No.: US 2026/0125459 A1
(43) Pub. Date: May 7, 2026

(54) Macrophage depletion and vesicular stomatitis virus for treatment of cancer

(71) Applicant: Boehringer Ingelheim International GmbH, Ingelheim am Rhein (DE)

(72) Inventors: Melissa MAYR, Imsterberg (AT); Monika PETERSSON, Eisingen (DE)

(21) Appl. No.: 19/357,131

(22) Filed: Oct. 14, 2025

(30) Foreign Application Priority Data
Oct. 15, 2024 (EP) 24206598.5

(51) Int. Cl. C07K 16/24; A61K 35/766; A61K 38/16; A61P 35/00; C07K 16/28

(52) U.S. Cl. CPC C07K 16/243; A61K 35/766; A61K 38/162; A61P 35/00; C07K 16/2866; C07K 2317/76

(57) ABSTRACT

The present invention relates to the treatment of cancers and in particular to the treatment of cancers with a macrophage depleting agent and a recombinant vesicular stomatitis virus.

### RELATED APPLICATIONS

This application claims the benefit of European Patent Application No. EP 24206598.5, filed Oct. 15, 2024.

### SEQUENCE LISTING

The instant application contains a Sequence Listing which has been submitted electronically in XML format and is hereby incorporated by reference in its entirety. Said XML copy, created on Aug. 25, 2025, is named 01-3606-US-1_SL.xml and is 16,085 bytes in size.

### FIELD OF THE INVENTION

The present invention relates to the treatment of cancers and in particular to the treatment of cancers with a macrophage depletion agent and a recombinant vesicular stomatitis virus.

### BACKGROUND OF THE INVENTION

The rationale to target macrophages, either by repolarization or depletion, to fight cancer exists since 1985. Macrophages are well known to play a pivotal role in anti-viral defense mechanisms. In general, macrophages within the tumor are impacting immune surveillance due to impacting anti-tumor immune cell activity and creating an immune suppressive environment. Among different agents that have been subjected to clinical trials, it has been shown that CSF1R inhibition preferentially eliminates M2 immunosuppressive macrophage and resulted in a change in the ratio of CD8/CD4 T-cell levels, indicating a conversion towards a more immuno-competent TME.

Wild-type vesicular stomatitis virus (VSV) is capable to infect macrophages, especially sub-capsular sinus macrophages within the spleen. It has been well established that these viruses can replicate within these macrophages for one or two cycles. In turn, macrophages can stimulate type I Interferon signalling, thereby blocking virus replication and limiting viral spread. Corroborating findings have been reported for the capability of VSV-GP (a recombinant VSV expressing the glycoprotein of LCMV instead of the wild-type glycoprotein) to transiently infect specialized CD169+ve macrophage populations within the spleen and liver. However, little is known about the role of macrophages and VSV-GP replication within in the tumor.

### SUMMARY OF THE INVENTION

It is to be understood that any embodiment relating to a specific aspect might also be combined with another embodiment also relating to that specific aspect, even in multiple tiers and combinations comprising several embodiments to that specific aspect.

The present invention provides in a first aspect, a method of treating cancer in a subject, comprising administering a macrophage depleting agent to the subject and thereby treating the cancer.

In an embodiment, relating to the first aspect, the cancer is a solid cancer. In a related embodiment, the cancer is selected from the list consisting of: a reproductive tumor, an ovarian tumor, a testicular tumor, an endocrine tumor, a gastrointestinal tumor, a pancreatic tumor, a liver tumor, a kidney tumor, a colon tumor, a colorectal tumor, a bladder tumor, a prostate tumor, a skin tumor, melanoma, a respiratory tumor, a lung tumor, a breast tumor, a head & neck tumor, a head and neck squamous-cell carcinoma (HNSCC) and a bone tumor.

In an embodiment, relating to the first aspect or any of its embodiments, the method further comprises administering a recombinant vesicular stomatitis virus to the subject. In a related embodiment, the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV. In a further related embodiment, the recombinant vesicular stomatitis virus is administered concomittantly, sequentially or alternately with the macrophage depleting agent.

In an embodiment, relating to the first aspect or any of its embodiments, the macrophage depleting agent is a colony-stimulating factor receptor (CSFR) inhibitor. In a related embodiment, the CSFR inhibitor blocks CSFR on tumor associated macrophages. In a further related embodiment, the CSFR inhibitor is an antagonistic antibody which is directed against CSFR, preferably CSF1R.

In another embodiment, the CSFR inhibitor is selected from the group consisting of ataxilimab, emactuzumab, cabiralizumab, pexidartinib, edicotinib, sotuletinib, ARRY-382, AMG820, GW2580, PLX7486, Ki20227, LY3022855 IMC-CS4, PLX3397 or PLX5622.

In an embodiment, relating to the first aspect or any of its embodiments, the macrophage depleting agent is a colony-stimulating factor 1 (CSF1) inhibitor. In a related embodiment, the CSF1 inhibitor is an antagonistic antibody which is directed against CSF1.

In a second aspect, the invention relates to a composition comprising a macrophage depleting agent and a recombinant vesicular stomatitis virus.

In a third aspect, the invention relates to a kit of parts comprising:
a) a recombinant vesicular stomatitis virus or a pharmaceutical composition comprising said recombinant vesicular stomatitis virus, and
b) a macrophage depleting agent.

It will be understood that any of the herein disclosed methods also equally apply to the use of any of the disclosed macrophage depleting agents alone or in combination with a recombinant vesicular stomatitis virus, in such methods, e.g., a macrophage depleting agent alone or in combination with a recombinant vesicular stomatitis virus, as disclosed herein, for use in the treatment, prevention, reducing and/or amelioration of any of the disclosed diseases and/or conditions. In other words, the invention also provides for the use of a macrophage depleting agent alone or in combination with a recombinant vesicular stomatitis virus, as disclosed herein, for the manufacture of a medicament for the treatment, prevention, reducing and/or amelioration of any of the disclosed diseases and/or conditions.

### BRIEF DESCRIPTION OF THE DRAWINGS

FIGS. 1A-B Human HNSCCs are susceptible to VSV-GP ex vivo. (A) Following vibratome sectioning of fresh patient-derived human HNSCC samples, slices were treated with a GFP-tagged VSV-GP to follow virus kinetics. GFP signal was analyzed via live microscopy over time (24 and 48 h). (B) Live captures of VSV-GP-GFP infected slices 24 and 48 h post treatment. Brightfield and GFP (VSV-GP-GFP) pictures of a non-permissive and permissive case are shown.

FIGS. 2A-D Macrophages may inhibit viral spread in human HNSCCs ex vivo. (A) Live captures of a VSV-GP-GFP infected patient-derived HNSCC slice 24 and 48 h post treatment. White arrows mark the not infected central area surrounded by infected cells (ring-like pattern). DAPI was used as nuclear counterstain. (B) Immunofluorescence stainings using αVSV-N and αCD68 antibodies on FFPE sections of a VSV-GP treated patient-derived HNSCC slice. DAPI was used as nuclear counterstain. (C) Cytokines in the supernatant of VSV-GP-GFP infected patient-derived HNSCC slices (n=3) were analyzed 48 hours post infection (hpi). Isotype control (IgG) treated slices served as controls. Measured values were normalized to the IgG control. (D) Secreted cytokines of VSV-GP-GFP infected HNSCC slices (n=12) at 48 hpi were normalized to the IgG control and divided into non-, semi- and highly-permissive tumors.

FIGS. 3A-B αCSF-1R treatment depletes F4/80+ cells in MC38 tumors. C57/BL6 were inoculated s.c. with 5×10⁵ MC38 cells. Mice were treated with 1 mg of either isotype control or αCSF-1R i.p. three times a week or not treated at all. 3 and 8 days after treatment start tumors were and either used for FACS analysis or fixed with formalin for histophatological analysis. (A) Untreated, isotype control or αCSF-1R treated MC38 tumors (n=5) were harvested 3 and 8 days post treatment start. FACS analysis was performed on these tumors. The percentage of F4/80+ cell in the myeloid population (CD11b+) is shown. (B) Representative immunofluorescence stainings using F4/80 on FFPE sections of αCSF-1R or isotype control treated MC38 tumors. DAPI was used as nuclear counterstain.

FIGS. 4A-E In vivo αCSF-1R treatment increases viral spread in murine tumor slice cultures ex vivo. (A) C57/BL6 were inoculated s.c. with 5×10⁵ MC38 cells. Mice were treated with 1 mg of either isotype control or αCSF-1R i.p. three times a week starting at day 10. On day 28 tumors were harvested, sectioned and treated with VSV-GP-GFP ex vivo (1×10⁵ TCID₅₀). Mock treated slices served as a control. (B) Live captures of mock or VSV-GP-GFP infected slices 24 h and 48 h post treatment following ex vivo treatment. (C) Tumor slices were scored with a 5-tiered scoring system depending on the amount of GFP positive cells 14, 24 and 48 h post infection. The graph displaying the GFP-Scores for the 2 in vivo treatments are shown. (D) Genomic copies of VSV-N were measured in infected slices 24 and 48 hpi via qPCR. The quantification data are shown. € Whole mount immunofluorescent staining on VSV-GP-GFP infected murine tumor slices 24 and 48 hpi using a VSV-N antibody. DAPI was used as nuclear counterstain.

FIGS. 5A-C Synergistic effects of αCSF-1R and VSV-GP in MC38 tumors. (A) C57/BL6 were inoculated s.c. with 5×10⁵ MC38 cells. Mice were treated with 1 mg of either isotype control or αCSF-1R i.p. three times a week starting at day 5 until day 21. On day 13 tumors were treated i.t. with 1×10⁸ TCID₅₀ VSV-GP or vehicle control. 5 tumors per group were harvested on day 16 for immunohistochemistry analysis. (B) Tumor growth of the remaining MC38 tumors was monitored up to 94 days. Tumor volume graphs are shown. (C) Kaplan-Meier survival curve is shown.

FIGS. 6A-B Prolonged viral replication in αCSF-1R treated MC38 tumors. (A) Immunofluorescence stainings using a αVSV-N antibody on FFPE sections of VSV-GP treated tumors (day 14, d16 and d20), +/−αCSF-1R pre-treatment, were performed to investigate viral spread. DAPI was used as nuclear counterstain. (B) Interferon-α and β levels in tumors were measured using the MSD multiplex system.

### DETAILED DESCRIPTION OF THE INVENTION

The present invention is based on the finding that in human tumor slice cultures certain slice culture tissues were less permissive to ex vivo infection with VSV-GP than other slice cultures. It was observed that in non-permissive or poorly permissive slice cultures ring-like pattern of GFP positive virus progeny formed around cell clusters. Those ring-like patterns represented cells that were initially infected with VSV-GP, but propagation and virus spread was restricted. After further investigation it was found that macrophages localized in close proximity to these infected cells in these ring-like structures. This prompted further investigations into the cytokine profiles of non-permissive, semi-permissive and highly permissive cases. Non- and semi-permissive cases showed higher secretion of cytokines associated with myeloid cell lineages compared to the highly permissive slice cultures. These experiments indicated that macrophages within these human tumor ecosystems could hamper oncolytic virus spread.

It was hypothesized that macrophage depletion and VSV-GP based oncolytic virus therapy could act together to improve cancer treatment. To this end tumor-bearing mice were treated with an antagonistic CSF1R antibody in combination with VSV-GP. Surprisingly, mice treated with the combination showed long-lasting and complete remissions following anti CSF1R and VSV-GP treatment. Thus, these data point to a synergistic effect of CSF1R blockage and VSV-GP, thereby pinpointing to a substantial role of macrophages depletion in facilitating virus spread.

In the following detailed description, numerous specific details are set forth to provide a full understanding of the present invention. It will be apparent, however, to one ordinarily skilled in the art that the subject technology may be practiced without some of these specific details. In other instances, well-known structures and techniques have not been shown in detail so as not to obscure the present invention. The headings are included merely for convenience to assist in reading and shall not be understood to limit the invention to specific aspects or embodiments.

### CSF1R

Colony stimulating factor 1 receptor (CSF1R), also known as macrophage colony-stimulating factor receptor (M-CSFR), and CD115 (Cluster of Differentiation 115), is a cell-surface protein encoded by the human CSF1R gene (known also as c-FMS). CSF1R is a receptor that can be activated by two ligands: colony stimulating factor 1 (CSF1) and interleukin-34 (IL-34). CSF1R is highly expressed in myeloid cells, and CSF1R signaling is necessary for the survival, proliferation, and differentiation of many myeloid cell types in vivo and in vitro.

CSF1R, the protein encoded by the CSF1R gene is a tyrosine kinase transmembrane receptor and member of the CSF1/PDGF receptor family of tyrosine-protein kinases. CSF1R has 972 amino acids, is predicted to have a molecular weight of 107.984 kilodaltons and is composed of an extracellular and a cytoplasmic domain. The extracellular domain has 3 N-terminal immunoglobulin (Ig) domains (D1-D3) which bind ligand, 2 Ig domains (D4-D5) which stabilize the ligand, a linker region, and a single-pass transmembrane helix. The cytoplasmic domain has a juxtamembrane domain and tyrosine kinase domain that is interrupted by a kinase insert domain. At rest, the juxtamembrane domain of CSF1R enters an autoinhibitory position to prevent signaling of the CSF1R cytosolic domain. Upon binding of ligand to extracellular Ig domains, CSF1R dimerizes noncovalently and autophosphorylates several tyrosine residues. This first wave of CSF1R tyrosine phosphorylation creates phosphotyrosine-binding domains to which effector proteins can bind and initiate various cellular responses. The first wave of tyrosine phosphorylation also leads to the covalent dimerization of CSF1R via disulfide bonds. Covalent CSF1R dimerization is important for a series of modifications to CSF1R itself including a second wave of tyrosine phosphorylation, serine phosphorylation, ubiquitination, and eventually endocytosis which terminates signaling by trafficking the ligand-CSF1R complex to the lysosome for degradation. Colony stimulating factor 1 (CSF1) and interleukin-34 (IL-34) are both CSF1R ligands. Both ligands regulate myeloid cell survival, proliferation, and differentiation, but CSF1 and IL-34 differ in their structure, distribution in the body, and the specific cellular signaling cascades triggered upon binding to CSF1R.

Monocytes and macrophages are mononuclear phagocytes. Monocytes circulate in the blood and are capable of differentiating into macrophages or dendritic cells, and macrophages are terminally differentiated tissue-resident cells. In macrophages whose survival is fully or partially dependent on CSF1R signaling, CSF1R promotes survival by activating PI3K. CSF1R signaling also regulates macrophage function. Activation of CSF1R is a strong chemokinetic signal, inducing macrophage polarization and chemotaxis towards the source of CSF1R ligand. This macrophage response requires rapid morphological changes which is achieved by remodeling of the actin cytoskeleton via the Src/Pyk2 and PI3K signaling pathways.

The canonical sequence of CSF1R is as shown below:

(SEQ ID NO: 1)
MGPGVLLLLLVATAWHGQGIPVIEPSVPELVVKPGATVTLRCVGNGSVE
WDGPPSPHWTLYSDGSSSILSTNNATFQNTGTYRCTEPGDPLGGSAAIH
LYVKDPARPWNVLAQEVVVFEDQDALLPCLLTDPVLEAGVSLVRVRGRP
LMRHTNYSFSPWHGFTIHRAKFIQSQDYQCSALMGGRKVMSISIRLKVQ
KVIPGPPALTLVPAELVRIRGEAAQIVCSASSVDVNFDVFLQHNNTKLA
IPQQSDFHNNRYQKVLTLNLDQVDFQHAGNYSCVASNVQGKHSTSMFFR
VVESAYLNLSSEQNLIQEVTVGEGLNLKVMVEAYPGLQGFNWTYLGPFS
DHQPEPKLANATTKDTYRHTFTLSLPRLKPSEAGRYSFLARNPGGWRAL
TFELTLRYPPEVSVIWTFINGSGTLLCAASGYPQPNVTWLQCSGHTDRC
DEAQVLQVWDDPYPEVLSQEPFHKVTVQSLLTVETLEHNQTYECRAHNS
VGSGSWAFIPISAGAHTHPPDEFLFTPVVVACMSIMALLLLLLLLLLYK
YKQKPKYQVRWKIIESYEGNSYTFIDPTQLPYNEKWEFPRNNLQFGKTL
GAGAFGKVVEATAFGLGKEDAVLKVAVKMLKSTAHADEKEALMSELKIM
SHLGQHENIVNLLGACTHGGPVLVITEYCCYGDLLNFLRRKAEAMLGPS
LSPGQDPEGGVDYKNIHLEKKYVRRDSGFSSQGVDTYVEMRPVSTSSND
SFSEQDLDKEDGRPLELRDLLHFSSQVAQGMAFLASKNCIHRDVAARNV
LLTNGHVAKIGDFGLARDIMNDSNYIVKGNARLPVKWMAPESIFDCVYT
VQSDVWSYGILLWEIFSLGLNPYPGILVNSKFYKLVKDGYQMAQPAFAP
KNIYSIMQACWALEPTHRPTFQQICSFLQEQAQEDRRERDYTNLPSSSR
SGGSGSSSSELEEESSSEHLTCCEQGDIAQPLLQPNNYQFC.

A macrophage depleting agent is an agent that reduces the numbers of macrophages in a tumor. Said reduction of tumor associated macrophages may be demonstrated by FACS analysis or histopathological analysis by counting the F4/80 positive macrophage population in a tumor model, e.g. in a MC38 murine tumor tissue model. An exemplary assay set up is described in the examples. Preferably, treatment of the tumor model with the macrophage depleting agent reduces the number of F4/80 positive macrophages in the tumor tissue compared to an untreated control by at least about 10%, 15%, 20%, 25%, 30%, 35%, 40%, 45% or by at least about 50%.

A CSF1R inhibitor is an agent that binds specifically to CSF1R and thereby competes with CSF1 ligand for binding to the CSF1R receptor (Joost et al. Multidimensional Profiling of CSF1R Screening Hits and Inhibitors: Assessing Cellular Activity, Target Residence Time, and Selectivity in a Higher Throughput Way; SLAS Discovery 2011; DOI: 10.1177/1087057111418113; Ries, Carola H. et al. Targeting Tumor-Associated Macrophages with Anti-CSF-1R Antibody Reveals a Strategy for Cancer Therapy; Cancer Cell 2014; DOI: 10.1016/j.ccr.2014.05.016; Liu et al. The mechanism of shared but distinct CSF-1R signaling by the non-homologous cytokines IL-34 and CSF-1; Biochimica et Biophysica Acta (BBA)—Proteins and Proteomics, 2012; DOI: 10.1016/j.bbapap.2012.04.012.). Preferably, the agent binding specifically to CSF1R has at least a 10-fold higher binding affinity to CSF1R, preferably to CSF1R as shown in SEQ ID NO:1, than to any other target or receptor.

In particular embodiments, the CSF1R inhibitor is a monoclonal antagonist antibody. Preferentially, said monoclonal antibody partially or fully blocks or inhibits a biological activity of a polypeptide or cell to which it specifically or preferentially binds, i.e. a CSF1R-expressing cells, more preferably a CSF1R-expressing human cells, and advantageously a CSF1R-expressing human cancer cells.

In another embodiment, the CSF1R inhibitor is a monoclonal antagonist antibody that either binds to CSF1 or to CSF1R and thereby reduces CSF1R dimerization or prevents CSF1R from dimerization compared to an untreated or mock treated control (Ries, Carola H. et al. Targeting Tumor-Associated Macrophages with Anti-CSF-1R Antibody Reveals a Strategy for Cancer Therapy; Cancer Cell 2014; DOI: 10.1016/j.ccr.2014.05.016). Preferably, dimerization is thereby reduced by at least 10%, 15%, 20%, 25%, 30%, 35%, 40%, 45%, or by at least 50% compared to the untreated or mock treated control.

Antagonistic monoclonal antibodies can be generated according to well-known methodologies-see for example Carola H. Ries et al., Targeting Tumor-Associated Macrophages with Anti-CSF-1R Antibody Reveals a Strategy for Cancer Therapy, Cancer Cell, Volume 25, Issue 6, 2014, Pages 846-859, ISSN 1535-6108. Briefly, mice may be immunized with an expression vector encoding the extra cellular domain of huCSF1R. Splenocyte-derived hybridomas may then be subcloned and screened for inhibition of receptor-ligand interaction by ELISA and inhibition of phosphorylation in human wild-type CSF1R overexpressing NIH 3T3 cells.

Recently, several CSF1R inhibitors were reviewed by Jiachen Wen et al., CSF1R inhibitors are emerging immunotherapeutic drugs for cancer treatment, European Journal of Medicinal Chemistry, Volume 245, Part 1, 2023, 114884, ISSN 0223-5234.

In one embodiment, the CSF1R inhibitor is PLX3397 (pexidartinib).

In one embodiment, the CSF1R inhibitor is HMPL-012 (sulfatinib).

In one embodiment, the CSF1R inhibitor is PLX5622.

In one embodiment, the CSF1R inhibitor is PLX73086.

In one embodiment, the CSF1R inhibitor is PLX7486.

In one embodiment, the CSF1R inhibitor is JNJ-40346527 (edicotinib).

In one embodiment, the CSF1R inhibitor is BLZ945 (sotuletinib).

In one embodiment, the CSF1R inhibitor is ARRY-382 (PF-07265804).

In one embodiment, the CSF1R inhibitor is ABT-869 (linifanib).

In one embodiment, the CSF1R inhibitor is DCC-3014 (vimseltinib).

In one embodiment, the CSF1R inhibitor is CS2164 (chiauranib).

In one embodiment, the CSF1R inhibitor is 3D185.

In one embodiment, the CSF1R inhibitor is NMS-03592088.

In one embodiment, the CSF1R inhibitor is QSI-930.

In one embodiment, the CSF1R inhibitor is Q702.

In one embodiment, the CSF1R inhibitor is TPX-0022 (elzovantinib).

In one embodiment, the CSF1R inhibitor is ARQ087 (derazantinib).

In one embodiment, the CSF1R inhibitor is X-82 (vorolanib).

In one embodiment, the CSF1R inhibitor is RG-7155.

### Vesicular Stomatitis Virus

Characterizing features of the vesicular stomatitis virus includes one or more of the following: A bullet-shaped or bacilliform particle 100-430 nm in length and 45-100 nm in diameter comprised of a helical nucleocapsid surrounded by a matrix layer and a lipid envelope. A negative-sense, single-stranded RNA of 10.8-16.1 kb, which are mostly unsegmented. A genome encoding for at least 5 genes encoding the structural proteins nucleoprotein (N), large protein (L), phosphoprotein (P), matrix protein (M), and glycoprotein (G).

Preferably, the recombinant vesicular stomatitis virus of the invention is an oncolytic virus. In this respect, oncolytic has its regular meaning known in the art and refers to the ability of a vesicular stomatitis virus to infect and lyse (break down) cancer cells but not normal cells (to any significant extend). Preferably, the oncolytic vesicular stomatitis virus is capable of replication within cancer cells. Oncolytic activity may be tested in different assay systems known to the skilled artisan (an exemplary in vitro assay is described by Muik et al., Cancer Res., 74 (13), 3567-78, 2014). It is to be understood that an oncolytic vesicular stomatitis virus may infect and lyse only specific types of cancer cells. Also, the oncolytic effect may vary depending on the type of cancer cells.

The term “recombinant” refers to a virus, more particularly a vesicular stomatitis virus, comprising an exogenous nucleic acid sequence inserted in its genome, which is not naturally present in the parent virus. A recombinant virus thus refers to a nucleic acid or virus made by an artificial combination of two or more segments of nucleic acid sequence of synthetic or semisynthetic origin which does not occur in nature or is linked to another nucleic acid in an arrangement not found in nature. The artificial combination is most commonly accomplished by artificial manipulation of isolated segments of nucleic acids, using well-established genetic engineering techniques. Generally, a “recombinant” vesicular stomatitis virus as described herein refers to virus that are produced by standard genetic engineering methods, e.g., vesicular stomatitis virus of the present invention are thus genetically engineered or genetically modified viruses. The term “recombinant vesicular stomatitis virus” thus includes viruses, which have stably integrated recombinant nucleic acid in their genome.

Preferred is the vesicular stomatitis virus (VSV) and in particular the VSV-GP (recombinant with GP of LCMV). Advantageous properties of the VSV-GP include one or more of the following: very potent and fast killer (<8 h); oncolytic virus; systemic application possible; reduced neurotropism/neurotoxicity; it reproduces lytically and induces immunogenic cell death; does not replicate in healthy human cells, due to interferon (IFN) response; strong activation of innate immunity; about 3 kb space for immunomodulatory cargos and antigens; recombinant with an arenavirus glycoprotein from the Lympho-Chorio-Meningitis-Virus (LCMV); favorable safety features in terms of reduced neurotoxicity and less sensitive to neutralizing antibody responses and complement destruction as compared to the wild type VSV (VSV-G); specifically replicates in tumor cells, which have lost the ability to mount and respond to anti-viral innate immune responses (e.g. type-I IFN signaling); abortive replication in “healthy cells” so is rapidly excluded from normal tissues; viral replication in tumor cells leads to the induction of immunogenic cell death, release of tumor associated antigens, local inflammation and the induction of anti-tumor immunity.

In a preferred embodiment the recombinant vesicular stomatitis virus encodes in its genome at least for a vesicular stomatitis virus nucleoprotein (N) comprising an amino acid sequence as set forth in SEQ ID NO:2 or a functional variant at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:2, a phosphoprotein (P) comprising an amino acid sequence as set forth in SEQ ID NO:3 or a functional variant at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:3, a large protein (L) comprising an amino acid sequence as set forth in SEQ ID NO:4 or a functional variant at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO:4, and a matrix protein (M) comprising an amino acid sequence as set forth in SEQ ID NO: 5 or a functional variant at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO: 5.

It is understood by the skilled artisan that modifications to the vesicular stomatitis virus nucleoprotein (N), large protein (L), phosphoprotein (P), matrix protein (M), or glycoprotein (G) sequence can be made without losing the basic functions of those proteins. Such functional variants as used herein retain all or part of their basic function or activity. The protein L for example is the polymerase and has an essential function during transcription and replication of the virus. A functional variant thereof must retain at least part of this ability. A good indication for retention of basic functionality or activity is the successful production of viruses, including these functional variants, that are still capable to replicate and infect tumor cells. Production of viruses and testing for infection and replication in tumor cells may be tested in different assay systems known to the skilled artisan (an exemplary in vitro assay is described by Muik et al., Cancer Res., 74 (13), 3567-78, 2014).

In a preferred embodiment the recombinant vesicular stomatitis virus encodes in its genome at least for a vesicular stomatitis virus nucleoprotein (N), large protein (L), phosphoprotein (P), matrix protein (M), glycoprotein (G), wherein the large protein (L) comprises an amino acid sequence having a sequence identity≥80% of SEQ ID NO:4.

In a preferred embodiment the recombinant vesicular stomatitis virus encodes in its genome at least for a vesicular stomatitis virus nucleoprotein (N), large protein (L), phosphoprotein (P), matrix protein (M), glycoprotein (G), wherein the nucleoprotein (N) comprises an amino acid sequence having a sequence identity≥90% of SEQ ID NO:2.

In a further preferred embodiment the recombinant vesicular stomatitis virus encodes in its genome at least for a vesicular stomatitis virus nucleoprotein (N), large protein (L), phosphoprotein (P), matrix protein (M), glycoprotein (G) and at least one GSDM or a functional variant thereof, wherein the large protein (L) comprises an amino acid sequence having a sequence identity equal or greater 80% of SEQ ID NO:4 and the nucleoprotein (N) comprises an amino acid sequence having a sequence identity≥90% of SEQ ID NO:2.

It is to be understood that a vesicular stomatitis virus of the invention may encode in its genome further cargos, such as tumor antigens, further chemokines, or other immunomodulatory elements.

In a further embodiment the recombinant vesicular stomatitis virus of the invention additionally encodes in its genome a sodium iodide symporter protein (NIS). Expression of NIS and co-incubation with e.g. ¹²⁵I allows the use of NIS as imaging reporter (Carlson et al., Current Gene Therapy, 12, 33-47, 2012).

TABLE 1

| Identifier | Sequence | SEQ ID NO: |
|---|---|---|
| VSV N | MSVTVKRIIDNTVVVPKLPANEDPVEYPADYFRKSKEIPLYI | 2 |
| | NTTKSLSDLRGYVYQGLKSGNVSIIHVNSYLYGALKDIRGKL | |
| | DKDWSSFGINIGKAGDTIGIFDLVSLKALDGVLPDGVSDAS | |
| | RTSADDKWLPLYLLGLYRVGRTQMPEYRKKLMDGLTNQC | |
| | KMINEQFEPLVPEGRDIFDVWGNDSNYTKIVAAVDMFFHM | |
| | FKKHECASFRYGTIVSRFKDCAALATFGHLCKITGMSTEDV | |
| | TTWILNREVADEMVQMMLPGQEIDKADSYMPYLIDFGLSS | |
| | KSPYSSVKNPAFHFWGQLTALLLRSTRARNARQPDDIEYT | |
| | SLTTAGLLYAYAVGSSADLAQQFCVGDNKYTPDDSTGGLT | |
| | TNAPPQGRDVVEWLGWFEDQNRKPTPDMMQYAKRAVMS | |
| | LQGLREKTIGKYAKSEFDK | |
| VSV P | MDNLTKVREYLKSYSRLDQAVGEIDEIEAQRAEKSNYELFQ | 3 |
| | EDGVEEHTKPSYFQAADDSDTESEPEIEDNQGLYAPDPEA | |
| | EQVEGFIQGPLDDYADEEVDVVFTSDWKQPELESDEHGKT | |
| | LRLTSPEGLSGEQKSQWLSTIKAVVQSAKYWNLAECTFEA | |
| | SGEGVIMKERQITPDVYKVTPVMNTHPSQSEAVSDVWSLS | |
| | KTSMTFQPKKASLQPLTISLDELFSSRGEFISVGGDGRMSH | |
| | KEAILLGLRYKKLYNQARVKYSL | |
| VSV L | MEVHDFETDEFNDFNEDDYATREFLNPDERMTYLNHADY | 4 |
| | NLNSPLISDDIDNLIRKENSLPIPSMWDSKNWDGVLEMLTS | |
| | CQANPIPTSQMHKWMGSWLMSDNHDASQGYSFLHEVDK | |
| | EAEITFDVVETFIRGWGNKPIEYIKKERWTDSFKILAYLCQK | |
| | FLDLHKLTLILNAVSEVELLNLARTFKGKVRRSSHGTNICRIR | |
| | VPSLGPTFISEGWAYFKKLDILMDRNFLLMVKDVIIGRMQTV | |
| | LSMVCRIDNLFSEQDIFSLLNIYRIGDKIVERQGNFSYDLIKM | |
| | VEPICNLKLMKLARESRPLVPQFPHFENHIKTSVDEGAKIDR | |
| | GIRFLHDQIMSVKTVDLTLVIYGSFRHWGHPFIDYYTGLEKL | |
| | HSQVTMKKDIDVSYAKALASDLARIVLFQQFNDHKKWFVN | |
| | GDLLPHDHPFKSHVKENTWPTAAQVQDFGDKWHELPLIKC | |
| | FEIPDLLDPSIIYSDKSHSMNRSEVLKHVRMNPNTPIPSKKV | |
| | LQTMLDTKATNWKEFLKEIDEKGLDDDDLIIGLKGKERELKL | |
| | AGRFFSLMSWKLREYFVITEYLIKTHFVPMFKGLTMADDLT | |
| | AVIKKMLDSSSGQGLKSYEAICIANHIDYEKWNNHQRKLSN | |
| | GPVFRVMGQFLGYPSLIERTHEFFEKSLIYYNGRPDLMRVH | |
| | NNTLINSTSQRVCWQGQEGGLEGLRQKGWSILNLLVIQRE | |
| | AKIRNTAVKVLAQGDNQVICTQYKTKKSRNVVELQGALNQ | |
| | MVSNNEKIMTAIKIGTGKLGLLINDDETMQSADYLNYGKIPIF | |
| | RGVIRGLETKRWSRVTCVTNDQIPTCANIMSSVSTNALTVA | |
| | HFAENPINAMIQYNYFGTFARLLLMMHDPALRQSLYEVQDK | |
| | IPGLHSSTFKYAMLYLDPSIGGVSGMSLSRFLIRAFPDPVTE | |
| | SLSFWRFIHVHARSEHLKEMSAVFGNPEIAKFRITHIDKLVE | |
| | DPTSLNIAMGMSPANLLKTEVKKCLIESRQTIRNQVIKDATIY | |
| | LYHEEDRLRSFLWSINPLFPRFLSEFKSGTFLGVADGLISLF | |
| | QNSRTIRNSFKKKYHRELDDLIVRSEVSSLTHLGKLHLRRG | |
| | SCKMWTCSATHADTLRYKSWGRTVIGTTVPHPLEMLGPQ | |
| | HRKETPCAPCNTSGFNYVSVHCPDGIHDVFSSRGPLPAYL | |
| | GSKTSESTSILQPWERESKVPLIKRATRLRDAISWFVEPDS | |
| | KLAMTILSNIHSLTGEEWTKRQHGFKRTGSALHRFSTSRMS | |
| | HGGFASQSTAALTRLMATTDTMRDLGDQNFDFLFQATLLY | |
| | AQITTTVARDGWITSCTDHYHIACKSCLRPIEEITLDSSMDY | |
| | TPPDVSHVLKTWRNGEGSWGQEIKQIYPLEGNWKNLAPA | |
| | EQSYQVGRCIGFLYGDLAYRKSTHAEDSSLFPLSIQGRIRG | |
| | RGFLKGLLDGLMRASCCQVIHRRSLAHLKRPANAVYGGLIY | |
| | LIDKLSVSPPFLSLTRSGPIRDELETIPHKIPTSYPTSNRDMG | |
| | VIVRNYFKYQCRLIEKGKYRSHYSQLWLFSDVLSIDFIGPFSI | |
| | STTLLQILYKPFLSGKDKNELRELANLSSLLRSGEGWEDIHV | |
| | KFFTKDILLCPEEIRHACKFGIAKDNNKDMSYPPWGRESRG | |
| | TITTIPVYYTTTPYPKMLEMPPRIQNPLLSGIRLGQLPTGAH | |
| | YKIRSILHGMGIHYRDFLSCGDGSGGMTAALLRENVHSRGI | |
| | FNSLLELSGSVMRGASPEPPSALETLGGDKSRCVNGETC | |
| | WEYPSDLCDPRTWDYFLRLKAGLGLQIDLIVMDMEVRDSS | |
| | TSLKIETNVRNYVHRILDEQGVLIYKTYGTYICESEKNAVTIL | |
| | GPMFKTVDLVQTEFSSSQTSEVYMVCKGLKKLIDEPNPDW | |
| | SSINESWKNLYAFQSSEQEFARAKKVSTYFTLTGIPSQFIPD | |
| | PFVNIETMLQIFGVPTGVSHAAALKSSDRPADLLTISLFYMAI | |
| | ISYYNINHIRVGPIPPNPPSDGIAQNVGIAITGISFWLSLMEK | |
| | DIPLYQQCLAVIQQSFPIRWEAVSVKGGYKQKWSTRGDGL | |
| | PKDTRISDSLAPIGNWIRSLELVRNQVRLNPFNEILFNQLCR | |
| | TVDNHLKWSNLRRNTGMIEWINRRISKEDRSILMLKSDLHE | |
| | ENSWRD | |
| VSV M | MSSLKKILGLKGKGKKSKKLGIAPPPYEEDTSMEYAPSAPID | 5 |
| | KSYFGVDEMDTYDPNQLRYEKFFFTVKMTVRSNRPFRTYS | |
| | DVAAAVSHWDHMYIGMAGKRPFYKILAFLGSSNLKATPAVL | |
| | ADQGQPEYHAHCEGRAYLPHRMGKTPPMLNVPEHFRRPF | |
| | NIGLYKGTIELTMTIYDDESLEAAPMIWDHFNSSKFSDFREK | |
| | ALMFGLIVEKKASGAWVLDSIGHFK | |
| LCMV GP | MGQIVTMFEALPHIIDEVINIVIIVLIIITSIKAVYNFATCGILALV | 6 |
| | SFLFLAGRSCGMYGLNGPDIYKGVYQFKSVEFDMSHLNLT | |
| | MPNACSANNSHHYISMGSSGLELTFTNDSILNHNFCNLTSA | |
| | FNKKTFDHTLMSIVSSLHLSIRGNSNHKAVSCDENNGITIQY | |
| | NLSFSDPQSAISQCRTFRGRVLDMFRTAFGGKYMRSGWG | |
| | WAGSDGKTTWCSQTSYQYLIIQNRTWENHCRYAGPFGMS | |
| | RILFAQEKTKFLTRRLAGTFTWTLSDSSGVENPGGYCLTK | |
| | WMILAAELKCFGNTAVAKCNVNHDEEFCDMLRLIDYNKAAL | |
| | SKFKQDVESALHVFKTTVNSLISDQLLMRNHLRDLMGVPY | |
| | CNYSKFWYLEHAKTGETSVPKCWLVTNGSYLNETHFSDQI | |
| | EQEADNMITEMLRKDYIKRQGSTPLALMDLLMFSTSAYLISI | |
| | FLHLVKIPTHRHIKGGSCPKPHRLTNKGICSCGAFKVPGVK | |
| | TIWKRR | |
| Dandenong GP | MGQLITMFEALPHIIDEVINIVIIVLVIITSIKAVYNFATCGIIALIS | 7 |
| | FCLLAGRSCGLYGVTGPDIYKGLYQFKSVEFNMSQLNLTM | |
| | PNACSANNSHHYISMGKSGLELTFTNDSIISHNFCNLTDGF | |
| | KKKTFDHTLMSIVASLHLSIRGNTNYKAVSCDFNNGITIQYN | |
| | LSFSDAQSAINQCRTFRGRVLDMFRTAFGGKYMRSGYGW | |
| | KGSDGKTTWCSQTSYQYLIIQNRTWENHCEYAGPFGLSRV | |
| | LFAQEKTKFLTRRLAGTFTWTLSDSSGTENPGGYCLTKWM | |
| | LIAAELKCFGNTAVAKCNINHDEEFCDMLRLIDYNKAALKKF | |
| | KEDVESALHLFKTTVNSLISDQLLMRNHLRDLMGVPYCNYS | |
| | KFWYLEHVKTGDTSVPKCWLVSNGSYLNETHFSDQIEQEA | |
| | DNMITEMLRKDYIKRQGSTPLALMDLLMFSTSAYLISVFLHL | |
| | MKIPTHRHIKGGTCPKPHRLTSKGICSCGAFKVPGVKTVWK | |
| | RR | |
| Mopeia GP | MGQIVTFFQEVPHILEEVMNIVLMTLSILAILKGIYNVMTCGII | 8 |
| | GLITFLFLCGRSCSSIYKDNYEFFSLDLDMSSLNATMPLSCS | |
| | KNNSHHYIQVGNETGLELTLTNTSIIDHKFCNLSDAHRRNLY | |
| | DKALMSILTTFHLSIPDFNQYEAMSCDFNGGKISIQYNLSHS | |
| | NYVDAGNHCGTIANGIMDVFRRMYWSTSLSVASDISGTQCI | |
| | QTDYKYLIIQNTSWEDHCMFSRPSPMGFLSLLSQRTRNFYI | |
| | SRRLLGLFTWTLSDSEGNDMPGGYCLTRSMLIGLDLKCFG | |
| | NTAIAKCNQAHDEEFCDMLRLFDFNKQAISKLRSEVQQSIN | |
| | LINKAVNALINDQLVMRNHLRDLMGIPYCNYSKFWYLNDTR | |
| | TGRTSLPKCWLVTNGSYLNETQFSTEIEQEANNMFTDMLR | |
| | KEYEKRQSTTPLGLVDLFVFSTSFYLISVFLHLIKIPTHRHIK | |
| | GKPCPKPHRLNHMAICSCGFYKQPGLPTQWKR | |

### Pseudotyped Vesicular Stomatitis Virus

It is known that wildtype VSV strains are considered to be neurotoxic. It is also reported that infected individuals are able to rapidly mount a strong humoral response with high antibody titers directed mainly against the glycoprotein. Neutralizing antibodies targeting the glycoprotein G of vesicular stomatitis virus are able to limit virus spread and thereby mediate protection of individuals from virus re-infection. Virus neutralization, however, limits repeated application of the virus to the cancer patient.

To eliminate these drawbacks the vesicular stomatitis virus wildtype glycoprotein G may be replaced with the glycoprotein from another virus. In this respect replacing the glycoprotein refers to (i) replacement of the gene coding for the wild type glycoprotein G with the gene coding for the glycoprotein GP of another virus, and/or (ii) replacement of the wild type glycoprotein G with the glycoprotein GP of another virus.

In a preferred embodiment the vesicular stomatitis virus glycoprotein G is replaced with the glycoprotein GP of the lymphocytic choriomeningitis virus (LCMV), preferably with the strain WE-HPI. In an even more preferred embodiment, the virus is a vesicular stomatitis virus with the glycoprotein GP of the lymphocytic choriomeningitis virus (LCMV), preferably with the strain WE-HPI. Such VSV is for example described in WO2010/040526 and named VSV-GP. Advantages offered are (i) the loss of VSV-G mediated neurotoxicity and (ii) a lack of vector neutralization by antibodies (as shown in mice).

The glycoprotein GP of the lymphocytic choriomeningitis virus (LCMV) may be GP1 or GP2. The invention includes glycoproteins from different LCMV strains. In particular, LCMV-GP can be derived from LCMV wild-type or LCMV strains LCMV-WE, LCMV-WE-HPI, LCMV-WE-HPI opt. In a preferred embodiment, the gene coding for the glycoprotein GP of the LCMV encodes for a protein with an amino acid sequence as shown in SEQ ID NO:6 or an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO:6 while the functional properties of the recombinant vesicular stomatitis virus comprising a glycoprotein GP encoding an amino acid sequence as shown in SEQ ID NO:6 are maintained.

In another embodiment the recombinant vesicular stomatitis virus glycoprotein G is replaced with the glycoprotein GP of the Dandenong virus (DANDV) or Mopeia (MOPV) virus. In a more preferred embodiment, the recombinant vesicular stomatitis virus is a vesicular stomatitis virus wherein the glycoprotein G is replaced with the glycoprotein GP of the Dandenong virus (DANDV) or Mopeia (MOPV) virus. Advantages offered are (i) the loss of VSV-G mediated neurotoxicity and (ii) a lack of vector neutralization by antibodies (as shown in mice).

The Dandenong virus (DANDV) is an old world arenavirus. To date, there is only a single strain known to the person skilled in the art, which comprise a glycoprotein GP and which may be employed within the present invention as donor of the glycoprotein GP comprised in the recombinant vesicular stomatitis virus. The DANDV glycoprotein GP comprised in the recombinant vesicular stomatitis virus has more than 6 glycosylation sites, in particular 7 glycosylation sites. An exemplary preferred glycoprotein GP is that as comprised in DANDV as accessible under Genbank number EU136038. In one embodiment, the gene coding for the glycoprotein GP of the DNADV encodes for an amino acid sequence as shown in SEQ ID NO:7 or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO:7 while the functional properties of the recombinant vesicular stomatitis virus comprising a glycoprotein GP encoding an amino acid sequence as shown in SEQ ID NO:7 are maintained.

The Mopeia virus (MOPV) is an old world arenavirus. There are several strains known to the person skilled in the art, which comprise a glycoprotein GP and which may be employed within the present invention as donor of the glycoprotein GP comprised in the recombinant vesicular stomatitis virus of the invention. The MOPV glycoprotein GP comprised in the recombinant vesicular stomatitis virus has more than 6 glycosylation sites, in particular 7 glycosylation sites. An exemplary preferred glycoprotein GP is that as comprised in Mopeia virus as accessible under Genbank number AY772170. In one embodiment, the gene coding for glycoprotein GP of the MOPV encodes for an amino acid sequence as shown in SEQ ID NO:8 or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO:8 while the functional properties of the recombinant vesicular stomatitis virus comprising a glycoprotein GP encoding an amino acid sequence as shown in SEQ ID NO:8 are maintained.

As used herein, the terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence. To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=#of identical positions/total #of positions (e.g., overlapping positions)×100). In some embodiments, the two sequences that are compared are the same length after gaps are introduced within the sequences, as appropriate (e.g., excluding additional sequence extending beyond the sequences being compared).

The determination of percent identity or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the XBLAST program, score=50, wordlength=3, to obtain amino acid sequences homologous to protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

In a preferred embodiment, a recombinant vesicular stomatitis virus encodes in its genome a vesicular stomatitis virus nucleoprotein (N), large protein (L), phosphoprotein (P), matrix protein (M), glycoprotein (G), wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV. In a related preferred embodiment,
the nucleoprotein (N) comprises an amino acid as set forth in SEQ ID NO: 2 or a functional variant at least 80%, 85%, 90%, 92%, 94%, 96%, 98% identical to SEQ ID NO:2
wherein the phosphoprotein (P) comprises an amino acid as set forth in SEQ ID NO:3 or a functional variant at least 80%, 85%, 90%, 92%, 94%, 96%, 98% identical to SEQ ID NO:3
wherein the large protein (L) comprises an amino acid as set forth in SEQ ID NO: 4 or a functional variant at least 80%, 85%, 90%, 92%, 94%, 96%, 98% identical to SEQ ID NO:4
the matrix protein (M) comprises an amino acid as set forth in SEQ ID NO: 5 or a functional variant at least 80%, 85%, 90%, 92%, 94%, 96%, 98% identical to SEQ ID NO:5.

### Combination Treatment

The present invention provides combination treatments/methods providing certain advantages compared to treatments/methods currently used and/or known in the prior art. These advantages may include in vivo efficacy (e.g. improved clinical response, extend of the response, increase of the rate of response, duration of response, disease stabilization rate, duration of stabilization, time to disease progression, progression free survival (PFS) and/or overall survival (OS), later occurrence of resistance and the like), safe and well tolerated administration and reduced frequency and severity of adverse events.

The recombinant vesicular stomatitis virus of the invention is used in combination treatment with a macrophage depletion agent. In one embodiment, the macrophage depleting agent is a colony-stimulating factor 1 (CSF1) inhibitor or a colony-stimulating factor receptor (CSFR) inhibitor. In another embodiment, the macrophage depleting agent is a colony-stimulating factor 1 receptor (CSF1R) inhibitor. In another embodiment, the CSF1 inhibitor or CSFR inhibitor is an antagonistic antibody which is directed against CSF1 or CSFR, respectively. In another embodiment, the CSFR inhibitor is an antagonistic antibody which is directed against CSF1R. In yet another embodiment, the inhibitor blocks CSF1 or CSFR on tumor associated macrophages.

Such a combined treatment may be given as a non-fixed (e.g. free) combination of the substances or in the form of a fixed combination, including kit-of-parts. In this context, “combination” or “combined” within the meaning of this invention includes, without being limited, a product that results from the mixing or combining of more than one active agent and includes both fixed and non-fixed (e.g. free) combinations (including kits) and uses, such as e.g. the simultaneous, concurrent, sequential, successive, alternate or separate use of the components or agents. The term “fixed combination” means that the active agents are both administered to a patient simultaneously in the form of a single entity or dosage. The term “non-fixed combination” means that the active agents are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active agents.

The invention provides for a recombinant vesicular stomatitis virus in combination with a macrophage depleting agent for use in the treatment of cancers as described herein, preferably for the treatment of solid cancers.

The invention also provides for the use of a recombinant vesicular stomatitis virus in combination with a macrophage depleting agent for the manufacture of a medicament for treatment and/or prevention of cancers as described herein, preferably for the treatment of solid cancers.

The invention further provides for a method for treating and/or preventing cancer, comprising administering a therapeutically effective amount of a recombinant vesicular stomatitis virus of the invention, and a macrophage depleting agent to an individual suffering from cancer, thereby ameliorating one or more symptoms of cancer. The recombinant vesicular stomatitis virus of the invention and the macrophage depleting agent may be administered concomitantly, sequentially or alternately.

The recombinant vesicular stomatitis virus of the invention and the macrophage depleting agent may be administered by the same administration routes or via different administration routes. The macrophage depleting agent may administered intravenously and the recombinant vesicular stomatitis virus of the invention may administered intratumorally. In another embodiment, the macrophage depleting agent may be administered intravenously and the recombinant vesicular stomatitis virus is administered at least once intratumorally and subsequent doses of the recombinant vesicular stomatitis virus are administered intravenously.

A combination as herein provided comprises (i) a recombinant vesicular stomatitis virus and (ii) a macrophage depleting agent, preferably an antagonistic antibody which is directed against CSF1R or CSF1. Further provided is the use of such a combination for the treatment of cancers as described herein.

In another aspect a combination treatment is provided comprising the use of (i) a recombinant vesicular stomatitis virus and (ii) a macrophage depleting agent. In such combination treatment the recombinant vesicular stomatitis virus may be administered concomitantly, sequentially or alternately with the macrophage depleting agent.

For example, “concomitant” administration includes administering the active agents within the same general time period, for example on the same day(s) but not necessarily at the same time. Alternate administration includes administration of one agent during a time period, for example over the course of a few days or a week, followed by administration of the other agent during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent during a second time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

Sequential treatment schedules include administration of the recombinant vesicular stomatitis virus followed by administration of the macrophage depleting agent. Sequential treatment schedules also include administration of the macrophage depleting agent followed by administration of the recombinant vesicular stomatitis virus of the invention. Sequential treatment schedules may include administrations 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or 31 days after each other.

In a preferred embodiment relating to the combination treatments the recombinant vesicular stomatitis virus encodes in its genome a vesicular stomatitis virus nucleoprotein (N), large protein (L), phosphoprotein (P), matrix protein (M), glycoprotein (G), wherein the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV.

### Pharmaceutical Compositions

The actual pharmaceutically effective amount or therapeutic dosage will of course depend on factors known by those skilled in the art such as age and weight of the patient, route of administration and severity of disease. In any case the recombinant vesicular stomatitis virus and the macrophage depleting agent will be administered at dosages and in a manner which allows a pharmaceutically effective amount to be delivered based upon patient's unique condition.

Generally, for the treatment and/or alleviation of the diseases, disorders and conditions mentioned herein and depending on the specific disease, disorder or condition to be treated, the potency of the recombinant vesicular stomatitis virus and the macrophage depleting agent to be used, the specific route of administration and the specific pharmaceutical formulation or composition used.

To be used in therapy, the recombinant vesicular stomatitis virus and the macrophage depleting agent of the invention are formulated (separately or together) into pharmaceutical compositions appropriate to facilitate administration to animals or humans. Typical formulations can be prepared by mixing the recombinant virus and/or the macrophage depleting agent with physiologically acceptable carriers, excipients or stabilizers, in the form of aqueous solutions or aqueous or non-aqueous suspensions. Carriers, excipients, modifiers or stabilizers are nontoxic at the dosages and concentrations employed. They include buffer systems such as phosphate, citrate, acetate and other inorganic or organic acids and their salts; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; proteins, such as serum albumin, gelatin, or immunoglobulins; hydrophilic polymers such as polyvinylpyrrolidone or polyethylene glycol (PEG); amino acids such as glycine, glutamine, asparagine, histidine, arginine, or lysine; monosaccharides, disaccharides, oligosaccharides or polysaccharides and other carbohydrates including glucose, mannose, sucrose, trehalose, dextrins or dextrans; chelating agents such as EDTA; sugar alcohols such as, mannitol or sorbitol; salt-forming counter-ions such as sodium; metal complexes (e.g., Zn-protein complexes); and/or ionic or non-ionic surfactants such as TWEEN™ (polysorbates), PLURONICS™ or fatty acid esters, fatty acid ethers or sugar esters. The excipients may also have a release-modifying or absorption-modifying function.

Depending on the type and severity of the disease, about 10⁶ to 10¹³ infectious particles measured by TCID₅₀ of the recombinant the recombinant vesicular stomatitis virus can be an initial candidate dosage for administration to the patient, whether, for example, by one or more separate administrations, or by continuous infusion. For repeated administrations over several days or longer, depending on the condition, the treatment would generally be sustained until a desired suppression of disease symptoms occurs. Thus, one or more doses of about 10⁶, 10⁷, 10⁸, 10⁹, 10¹⁰, 10¹¹, 10¹², or 10¹³ infectious particles measured by TCID₅₀ (or any combination thereof) may be administered to the patient. An initial higher loading dose, followed by one or more lower doses or vice versa may be administered. However, other dosage regimens may be useful. The progress of this therapy is easily monitored by conventional techniques and assays.

The actual pharmaceutically effective amount or therapeutic dosage will of course depend on factors known by those skilled in the art such as age and weight of the patient, route of administration and severity of disease. In any case the recombinant vesicular stomatitis virus and the macrophage depleting agent will be administered at dosages and in a manner which allows a pharmaceutically effective amount to be delivered based upon patient's unique condition.

An effective concentration of a recombinant vesicular stomatitis virus desirably ranges between about 10⁸ and 10¹⁴ vector genomes per milliliter (vg/mL). The infectious units may be measured as described in Mclaughlin et al., J Virol.; 62 (6): 1963-73 (1988). Preferably, the concentration is from about 1.5×10⁹ to about 1.5×10¹³, and more preferably from about 1.5×10⁹ to about 1.5×10¹¹. In one embodiment, the effective concentration is about 1.5×10⁹. In another embodiment, the effective concentration is about 1.5×10¹⁰. In another embodiment, the effective concentration is about 1.5×10¹¹. In yet another embodiment, the effective concentration is about 1.5×10¹². In another embodiment, the effective concentration is about 1.5×10¹³. In another embodiment, the effective concentration is about 1.5×10¹⁴. It may be desirable to use the lowest effective concentration in order to reduce the risk of undesirable effects. Still other dosages in these ranges may be selected by the attending physician, taking into account the physical state of the subject, preferably human, being treated, the age of the subject, the particular type of cancer and the degree to which the cancer, if progressive, has developed.

An effective target concentration of a recombinant vesicular stomatitis virus may be expressed with the TCID₅₀. The TCID₅₀ can be calculated for example by using the method of Spearman-Kärber. Desirably ranges include an effective target concentration between 1×10⁶/ml and 1×10¹⁴/ml TCID₅₀. Preferably, the effective target concentration is from about 1×10⁶ to about 1×10¹²/ml, and more preferably from about 1×10⁶ to about 1×10¹¹/ml. In one embodiment, the effective target concentration is about 1×10¹⁰/ml. In a preferred embodiment the target concentration is 5×10¹⁰/ml. In another embodiment, the effective target concentration is about 1.5×10¹¹/ml. In one embodiment, the effective target concentration is about 1×10¹²/ml. In another embodiment, the effective target concentration is about 1.5×10¹³/ml.

An effective target dose of a recombinant vesicular stomatitis virus may also be expressed with the TCID₅₀. Desirably ranges include a target dose between 1×10⁶ and 1×10¹⁴ TCID₅₀. Preferably, the target dose is from about 1×10⁶ to about 1×10¹³, and more preferably from about 1×10⁶ to about 1×10¹². In one embodiment, the effective concentration is about 1×10¹⁰. In a preferred embodiment, the effective concentration is about 1×10¹¹. In one embodiment, the effective concentration is about 1×10¹². In another embodiment, the effective concentration is about 1×10¹³.

An effective target dose of a CSFR1 inhibitor, preferably an antagonistic antibody, may be in the range of 0.1-100 mg/kg. In one aspect, the target dose of a CSFR1 inhibitor, preferably an antagonistic antibody, may be in the range of 0.3-20 mg/kg. In one aspect, a CSFR1 inhibitor, preferably an antagonistic antibody can be administered weekly or every two weeks via intravenous administration.

In another aspect, a kit or kit-of-parts containing materials useful for the treatment, prevention and/or diagnosis of the disorders described herein is provided. The kit or kit-of-parts comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the disorder and may have a sterile access port (for example the container may be an intravenous solution bag or a vial having a stopper pierceable by a hypodermic injection needle). At least one active agent in the composition is the recombinant vesicular stomatitis virus or pharmaceutical composition and one other active agent is the macrophage depleting agent. The label or package insert indicates that the composition is used for treating the condition of choice.

Moreover, the kit or kit-of-parts may comprise (a) a first container with a composition contained therein, wherein the composition comprises the recombinant vesicular stomatitis virus or pharmaceutical composition; and (b) a second container with a composition contained therein, wherein the composition comprises a macrophage depleting agent. The kit or kit-of-parts in this embodiment of the invention may further comprise a package insert indicating that the compositions can be used to treat a particular condition, in particular cancer. Alternatively, or additionally, the kit or kit-of-parts may further comprise a second (or third) container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

In a further aspect, a recombinant vesicular stomatitis virus is used in combination with a device useful for the administration of the recombinant virus, such as a syringe, injector pen, micropump, or other device. Preferably, a recombinant vesicular stomatitis virus is comprised in a kit of parts, for example also including a package insert with instructions for the use of the recombinant vesicular stomatitis virus.

### Medical Uses

A further aspect of the invention provides a recombinant vesicular stomatitis virus in combination with a macrophage depleting agent for use in medicine.

The recombinant vesicular stomatitis virus in combination with a macrophage depleting agent are useful for the treatment and/or prevention of cancer.

In a further aspect, the recombinant vesicular stomatitis virus in combination with a macrophage depleting agent can be used in a method for treating and/or preventing cancer, comprising administering a therapeutically effective amount of a recombinant vesicular stomatitis virus in combination with a macrophage depleting agent to an individual suffering from cancer, thereby ameliorating one or more symptoms of cancer.

In yet a further aspect the invention further provides for the use of a recombinant vesicular stomatitis virus in combination with a macrophage depleting agent for the manufacture of a medicament for treatment and/or prevention of cancer.

In yet a further aspect, the recombinant vesicular stomatitis virus in combination with a macrophage depleting agent can be used in a method for treating and/or preventing breast cancer, triple negative breast cancer, colorectal cancer, gastric cancer, gastrointestinal cancer, lung cancer or head & neck cancer, comprising administering a therapeutically effective amount of the recombinant vesicular stomatitis virus in combination with a macrophage depleting agent to an individual suffering from breast cancer, triple negative breast cancer, colorectal cancer, gastric cancer, gastrointestinal cancer, lung cancer or head & neck cancer, thereby ameliorating one or more symptoms of gastrointestinal cancer, lung cancer or head & neck cancer.

For the prevention or treatment of a disease, the appropriate dosage of the recombinant vesicular stomatitis virus in combination with a macrophage depleting agent will depend on a variety of factors such as the type of disease to be treated, as defined above, the severity and course of the disease, whether the recombinant vesicular stomatitis virus is administered for preventive or therapeutic purposes, previous therapy, the patient's clinical history and response, and the discretion of the attending physician. The recombinant vesicular stomatitis virus in combination with a macrophage depleting agent is suitably administered to the patient at one time or over a series of treatments.

In one aspect, the cancer is a solid cancer. The solid cancer may be reproductive cancer, ovarian cancer, testicular cancer, endocrine cancer, gastrointestinal cancer, pancreatic cancer, pancreatic adenocarcinoma, liver cancer, kidney cancer, colon cancer, colorectal cancer, bladder cancer, bladder urothelial carcinoma, muscle invasive bladder cancer (MIBC), non-muscle invasive bladder cancer (NMIBC), prostate cancer or carcinoma, skin cancer, (metastatic) melanoma, respiratory cancer, lung cancer, non-small cell lung cancer, small cell lung cancer, (metastatic) breast cancer or carcinoma, (metastatic) triple negative breast cancer (TNBC), head & neck cancer, head and neck squamous-cell carcinoma (HNSCC), bone cancer, gastric cancer, brain cancer, endometrial cancer, vaginal cancer, anal cancer, oropharyngeal squamous cell carcinoma, gastroesophageal junction adenocarcinoma, esophageal carcinoma, gastro esophageal junction (GEJ) cancer, oesophageal and gastroesophageal junction cancer, adenocarcinoma of the GEJ, hepatocellular carcinoma, cholangiocarcinoma, squamous cell carcinoma, and glioblastoma.

The recombinant vesicular stomatitis virus in combination with a macrophage depleting agent is administered by any suitable means, including oral, parenteral, subcutaneous, intratumoral, intravenous, intradermal, intraperitoneal, intrapulmonary, intracranial and intranasal. Parenteral infusions include intramuscular, intravenous, intraarterial, intraperitoneal, or subcutaneous administration. In addition, the recombinant vesicular stomatitis virus is suitably administered by pulse infusion. In one aspect, the dosing is given by injections, most preferably intravenous or subcutaneous injections, depending in part on whether the administration is brief or chronic.

The recombinant vesicular stomatitis virus may be administered via the same route or via different routes.

The term “suppression” is used herein in the same context as “amelioration” and “alleviation” to mean a lessening or diminishing of one or more characteristics of the disease.

Other features and advantages of the present invention will become apparent from the following more detailed Examples which illustrate, by way of example, the principles of the invention.

### EXAMPLES

### Methods

### Validation of Viral Replication (Fitness)—TCID₅₀/Cell Killing

HEK293F cells grown in suspension culture in Freestyle™ 293 Expression Medium (ThermoFisher Scientific) are infected with a low MOI (0.0005) of either VSV-GP or VSV-GP-GSDME-IL12. On the day of infection, the cells have a confluence of 60-70%. One well is counted (Countess™ cell counter, Invitrogen) before infecting the other wells with 0.005 MOI of one of the virus constructs. Culture supernatants (3 mL total volume) are harvested, and samples are analyzed 8 h, 16 h, 24 h, 32 h, 40 h and 48 h post infection for viral replication and cell killing. Viral replication is assessed using detection of viral genomes by qPCR in the supernatant of the cultures at the indicated timepoints. Virus induced cell killing is assessed by counting the viable cells in culture samples at the indicated time points.

### TCID₅₀ Assay

In 96-well plates 1×10⁴ BHK-21 cells in 100 μL supplemented GMEM (Gibco) are seeded per well. 24 h later, the adherent cells are infected with eleven 0.5×log 10 serial dilutions of the virus or the diluent alone (negative control) before incubation for three days at 37° C., 5% CO₂. Brightfield images of the cell culture wells are taken with the Tecan Spark Reader (Tecan) using a 4× objective. Whether the imaged wells are CPE positive or negative is assessed either by eye (i.e. visually) or automatically (i.e. via automated image analysis). The final TCID₅₀/mL is calculated by the formula of Spearman-Kärber (1,2). Six serial dilution replicates are assessed for each virus sample with each serial dilution on a separate plate for six plates total. Based on those six replicates the TCID₅₀/mL is calculated as described above.

Determination VSV-GP Genomic Copies by qPCR

RNA from cell culture supernatants is extracted using the MagMax-96 Viral RNA Isolation Kit (ThermoFisher, #AM1836) according to manufacturer's instructions. Genomic VSV-N copies are measured using iTaq Universal Probes One-Step Kit (BioRad, #1725141) with VSV-N primers (forward: 5′-AGT-ACC-GGA-GGA-TTG-ACG-ACT-AAT-3′, reverse: 5′-TCA-AAC-CAT-CCG-AGC-CAT-TC-3′) and probe (5′-ACC-GCC-ACA-AGG-CAG-AGA-TGT-GGT-3′). Amplification protocol: 50° C. for 10 min, 95° C. for 2 min and 40 cycles of 95° C. 15 s and 60° C. 30 s. A standard curve is set up by using VSV RNA in a concentration from 107 to 101 copies/ml. All qPCR samples are measured in technical triplicates.

### Flow Cytometric Analysis

Tumors are harvested three and eight days after αCSF-1R treatment. For dissociation of tumors, a combination of enzymatic digestion with murine tumor dissociation kit (Miltenyi Biotec) and mechanical dissociation on a OctoDissociator (program 37_mTDK_1) according to the manufacturer's instruction is used. Cells are then washed with PBS, cell pellet is suspended in a defined volume of PBS and stained with a live/dead discrimination dye. After blocking of Fc-receptors with a Fc-receptor blocking reagent (Biolegend), cell suspension from tumors are stained with antibodies characterizing macrophages.

The staining panels are as follows: CD11c Brilliant Violet 421 (clone REA754, Miltenyi Biotech), F4/80 Brilliant Violet 605 (clone BM8, BioLegend), CD45 FITC (clone REA737, Miltenyi Biotech), CD206 PE (clone C068C2, BioLegend), CD11b PerCP-Vio700 (clone REA592, Miltenyi Biotech), MHCII PE-Vio770 (clone REA813, Miltenyi Biotech), XCR1 APC (clone REA707, Miltenyi Biotech), Ly-6C AlexaFlour700 (clone HK1.4, BioLegend), CD90.2 APC-Vio770 (clone 30-H12, BioLegend), CD19 APC-Vio770 (clone REA749, Miltenyi Biotech), Ly-6G APC-Vio770 (clone REA526, Miltenyi Biotech), CD335 APC-Vio770 (clone REA815, Miltenyi Biotech) Counting beads (Invitrogen) are added to the final suspension and cells are analyzed on a MaxQuant flow cytometer (Miltenyi). Data are analyzed using FlowJo software and visualized using GraphPad Prism.

### In Vivo Experiments

Six to eight-week-old female mice are obtained from Janvier Labs (Le Genest-Saint-Isle, France) (Tumors are implanted by subcutaneous injection in the right flank. Tumor size is measured with a caliper and volume is calculated using the formula: tumor volume [mm³]=(length [mm])×(width [mm])²×0.5. Antibody treatment started A) when the mean tumor volume reaches a size of 80-150 mm³ B) 5 days after tumor implantation. Virus treatment started 8 days after the first antibody treatment. Antibody solutions are used for intraperitoneal (200 μl) injection. Virus solutions are used for intravenous (100 μl) or intratumoral (20 μl) injection. Mice are sacrificed when their tumor volume reach 1,500 mm³ or tumors show signs of ulcerations. Animals are euthanized by overdose on gas anesthesia (isoflurane) followed by cervical dislocation or exsanguination.

### Tumor Slice Culture Assay and Downstream Analysis

Sample collection. Following sample acquisition and immediate transport to the laboratory, tumor biospecimens are sectioned using a vibratome. Tissue slices are preserved just after sectioning (baseline sample) and submitted for pathological evaluation. Only samples with histologic features suggestive of tumor lesions and low numbers of dead cells are evaluated as adequate for subsequent analysis, retrospectively. Vibratome slices from different levels of the biospecimen are directly cultured for up to 48 h in an air-liquid interface or in a free-floating (FF) environment. Within the air-liquid interface, the specimen have access to the medium on the bottom and simultaneously to fresh oxygen on the top. This method provides ideal conditions for cultivating tumor tissue and study influences of oncolytic viruses on the tumor tissue. Within the free-floating culture, the specimen is fully surrounded by nutrients and medium, cytokines growth factors produced by the tissue are more homogeneously distributed and therefore is well suited to study immunomodulatory actions after virus infection. During the culturing period, virus replication is assessed by microscopy (Air-liquide interface) and cytokine secretion is tested (Free-floating). In addition, cultured samples are submitted for pathological evaluation by means of H&E and immunofluorescence studies are performed.

Histopathological assessment. Tumor slices are fixed in formalin, washed with phosphate-buffered saline (PBS) and embedded in Histogel (ThermoFisher Scientific #HG-400-012). Samples are then processed for paraffin embedding. Thin sections (4 μm) are cut with a rotating microtome (Thermo Scientific Microm HM 355S) with ˜10 sections per sample. Slides are stained with hematoxylin/eosin (HE) and assessed by a trained pathologist. Tumor cell numbers, cell numbers of the tumor microenvironment including immune cells, fibroblasts, endothelial cells and dead cells, which are still identifiable as cells, are quantified using digital pathology. Clinical biopsies of HNSCC may reveal a wide spectrum of pathohistological findings, only specimens with histologically confirmed tumor content and low numbers of dead cells are included in the analysis. Because the true nature of the specimen is unknown at the time the biopsy is taken, all samples not complying with the above criteria are retrospectively discarded.

Oncolytic virus treatment and permissivity testing. To receive a representative picture of the whole biospecimens, slices from different layers are allocated to different treatment groups and the respective baseline samples are preserved. All treatment arms comprise replicates from different layers of the biospecimen. Subsequently, tumor slices are treated with 1×10⁷ or 1×10⁵ TCID₅₀ VSV-GP or variants thereof. Mock treated slices serve as negative control. Samples are cultured at 37° C., 5% CO₂ in a humidified incubator. After an infection period of 24 to 48 h, tissue integrity and presence of the GFP signal within each single slice of the respective group is analyzed using a fluorescence microscope (Cell Observer, Zeiss). A five-tiered system is utilized to score for permissivity: 1-single GFP-positive cells or one small positive patch (<5 cells), 2-positive patch (around 20 cells) and few interspersed positive cells, 3-huge positive cluster (>50 cells), 4-multiple positive clusters/big patch and a lot of GFP-positive cells, 5-entire slice encompasses GFP-positive signal. Only cases displaying an intact morphology in the mock treated controls are included. A case is called permissive (=production of infectious progeny=GFP-positive) at 48 h post culturing, if more than half of the replicates score higher than 2. At 48 h, samples are fixed and further processed for paraffin embedding or frozen at −80° C. for viral copy number determination.

Microscopy and image processing. H&E stained slides are scanned using the Pannoramic Scan II (3D Histech) and pictures are exported using the Pannoramic Viewer Software. Immunofluorescence is captured using an inverted fluorescence microscope (Cell Observer, Zeiss).

Tissue lysis for qPCR analysis. Frozen tumor slices are thawed on ice and placed in a Lysis tube E (Analytic Jena) containing RLT lysis buffer. The tissue is lysed for 1 minute using a speedmill (Analytic Jena). After a short centrifugation the tissue lysate is used for RNA extraction and genomic copy determination.

Tissue lysis and cytokine analysis. Frozen tumor pieces are thawed on ice. 500 μl ProcartalPlex Cell Lysis buffer (Life Technologies) per 100 mg tissue are pipetted into a gentleMacs M tube and the tumor is transferred into the M tube. The tissue is lysed using a OctoDissociator (program Protein_01) according to the manufacturer's instruction. The tissue lysate is then centrifuged at 16000 g for 10 minutes at 4° C. and used for cytokine analysis using the MSD cytokine multiplex assay according to the manufacturer's instruction.

### Example 1

### Human HNSCCs are Susceptible to VSV-GP Ex Vivo

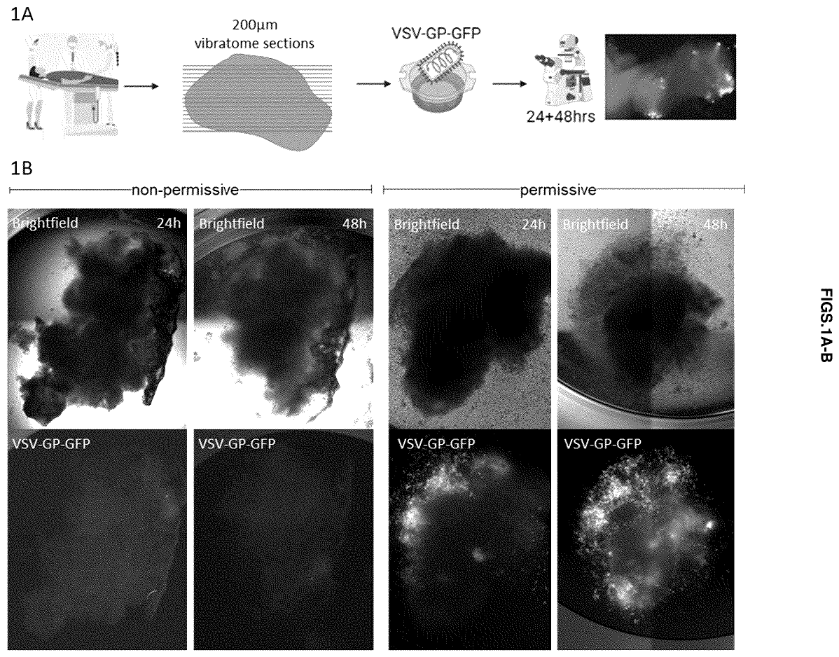

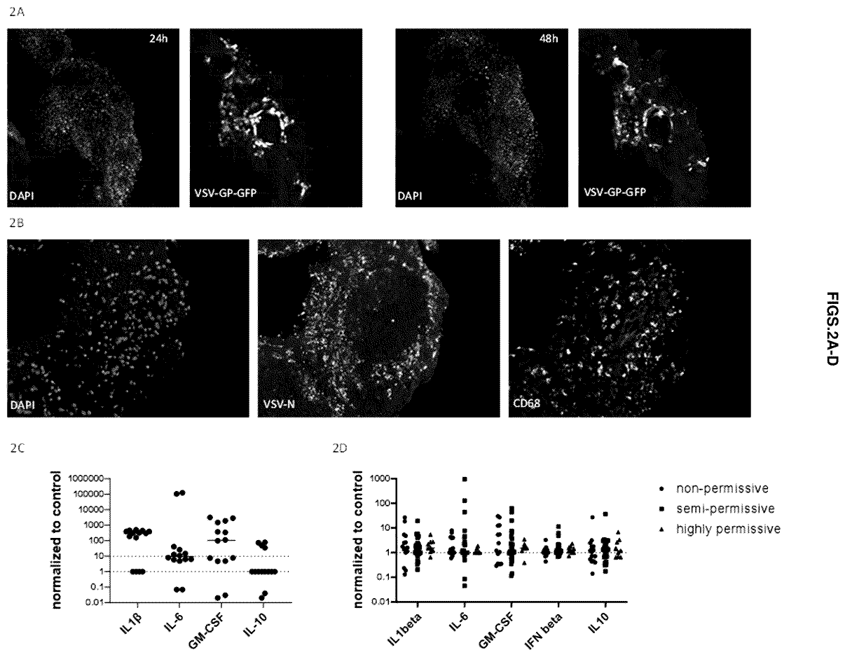

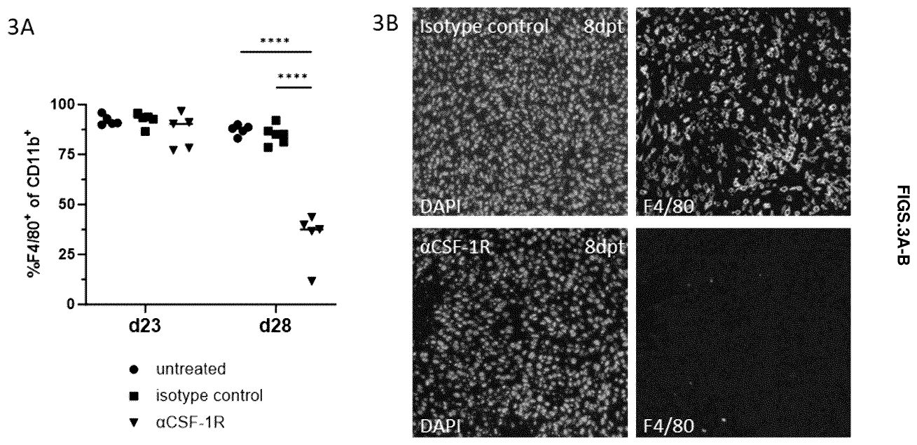

### FIGS. 1A-B & FIGS. 2A-D

Cancer evolves within a complex tissue environment consisting of diverse cell lineages such as T-cells, macrophages, dendritic cells, stromal cells, and non-cellular components (e.g. cytokines). These heterogeneous networks known as the tumor microenvironment (TME) influence tumor progression and suppression, thus affecting therapeutic success of immunomodulatory substances, e.g. oncolytic virotherapy. To understand permissivity and the mechanism of action of VSV-GP in the human tumor context, a patient-derived tumor slice culturing system for head and neck squamous cell carcinoma (HNSCC) was established. Within this 3D culturing approach of patient material, the polyclonal tumor architecture including components of the TME was preserved. In addition, we found that immune cells were still functional and could be activated. Thus, an immediate tumor-intrinsic immune response could be mounted within the HNSCCs ex vivo.

Next, we investigated whether patient-derived HNSCC slice cultures were susceptible to oncolytic virus infection (FIG. 1A). Here, we used VSV-GP-GFP, equipped with a GFP-tag to follow virus propagation and spread via live microscopy. GFP served as a read-out for permissivity, ascribing the potency of a virus not only to infect but also to produce infectious progeny. Intriguingly, we uncovered that the VSV-GP could not only infect but also propagate in 57% of the analyzed patient-derived HNSCC slice cultures (FIG. 1B). On a cellular level, VSV-GP could propagate within tumor and stromal compartment of the human ecosystems ex vivo and was able to induce apoptosis.

Besides highly permissive (GFP+) and non-permissive (GFP−) cases (FIG. 1B), frequently a ring-like pattern of GFP+ve virus progeny was observed, starting from 24 hpi, where the OV could initially infect, but propagation and virus spread was restricted (FIG. 2A). It was found that macrophages (CD68+ve) localized in close proximity to the infected N-positive cells, within the center of these ring-like structures (FIG. 2B). Interestingly, single double positive cells could be detected along the ring-like pattern 48 hpi. Furthermore, cytokines associated with myeloid cell lineages (IL6, GM-CSF, IL1b, IL10) were increased in supernatants of VSV-GP-GFP infected HNSCCs ex vivo (FIG. 2C). When looking at the cytokine profile of non-permissive, semi-permissive and highly permissive cases (n=12) similarities were observed in non- and semi-permissive cases, whereas the highly permissive ones showed lower secretion of cytokines associated with myeloid cell lineages (FIG. 2D).

In sum, these experiments indicated that macrophages within these human tumor ecosystems could hamper oncolytic virus spread.

### Example 2

### αCSF-1R Treatment Depletes F4/80+ Cells in MC38 Tumors.

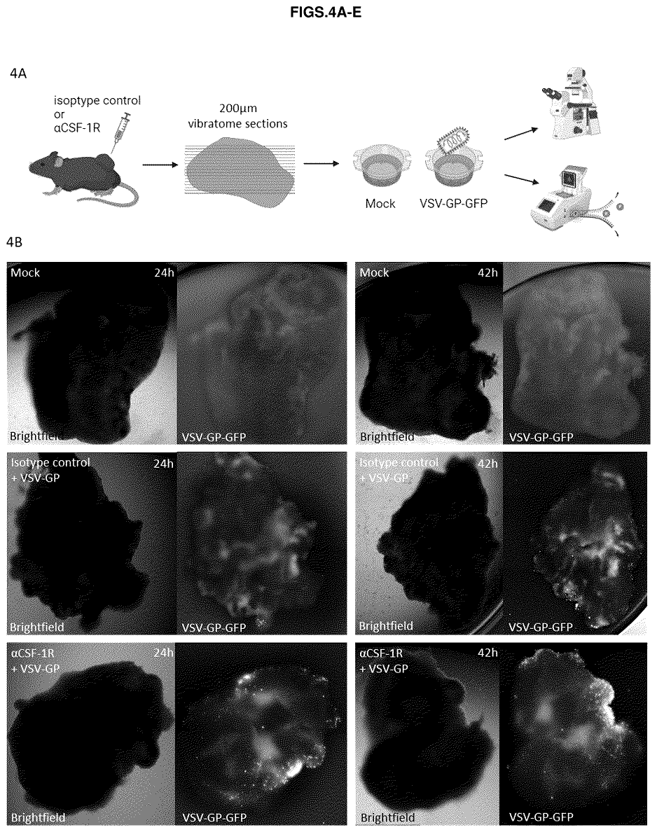

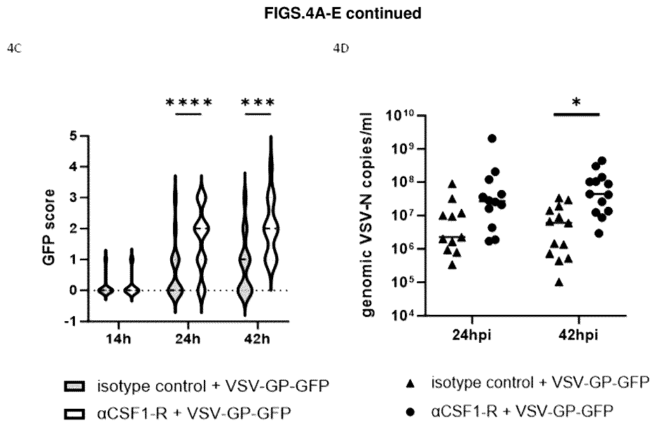

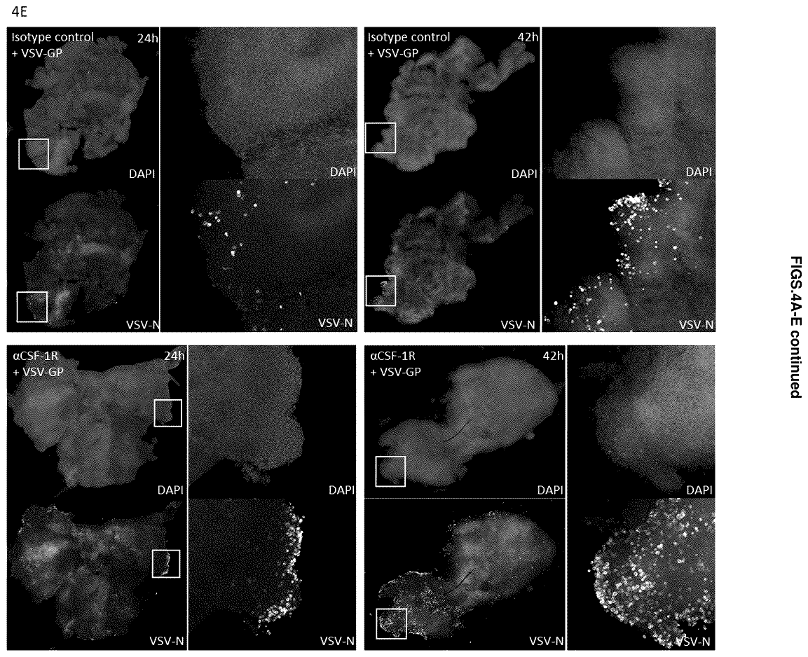

### FIGS. 3A-B/FIGS. 4A-E

To further investigate one the role of macrophages, in initial experiments, C57/BL6 were inoculated s.c. with 5×10⁵ MC38 cells. Mice were treated with 1 mg of either isotype control or αCSF-1R (AFS98 monoclonal antibody) i.p. three times a week or not treated at all. 3 and 8 days after treatment start tumors were either used for FACS analysis or fixed with formalin for histophatological analysis.

The AFS98 monoclonal antibody (Catalog #BE0213; Clone: AFS98; BioXcell) reacts with mouse colony stimulating factor 1 receptor (CSF1R), also known as macrophage colony-stimulating factor receptor (M-CSFR). CSF1R is a single-pass type I membrane protein and member of the platelet-derived growth factor receptor family. In mice CSF1R is expressed by monocytes/macrophages, peritoneal exudate cells, plasmacytoid and conventional dendritic cells, and osteoclasts. CSF1R is a receptor for CSF1 and CSF1 signaling through CSF1R regulates the proliferation and differentiation of cells in the monocytic lineage. The AFS98 antibody has been reported to deplete macrophages and block CSF1R in vivo.

α-CSF1-R treatment (1 mg i.p., 3×/week) in the MC38 murine tumor model over a time frame of 8 days resulted in a substantial decrease in F4/80 positive macrophage population as demonstrated by FACS analysis and immunofluorescence studies (FIG. 3A-B). This implies that the depletion of macrophages by α-CSF1-R treatment was successful and is in line with previous reports.

18 days following first treatment (treatment interval 3×/week), tumor tissue was harvested, sectioned and murine slice cultures were subsequently treated with VSV-GP-GFP (1×10⁵ TCID₅₀) ex vivo (FIG. 4A). Surprisingly, an increase in permissivity was observed in α-CSF1-R depleted tumor cultures (FIG. 4B) (as verified via N-immunofluorescence on whole-mounted tissue at 24 and 42 h after infection (FIG. 4E)) as well as a significantly faster virus spread starting already at 24 hpi (FIG. 4C). In contrast, a slower and limited virus spread was seen in VSV-GP-GFP infected tumor slice cultures that were treated with unspecific isotype control resulting in no macrophage depletion. Additional quantification of viral genomic copies in these infected slices showed again an increased viral replication in α-CSF1-R treated tumors (FIG. 4D).

Taken together, these studies showed increased viral spread and higher production of GFP+ve progeny ex vivo following α-CSF1-R depletion in vivo, indicating that macrophage depletion is resulting in improved VSV-GP spread within murine tumors.

### Example 3

### Synergistic Effects of αCSF-1R and VSV-GP in MC38 Tumors

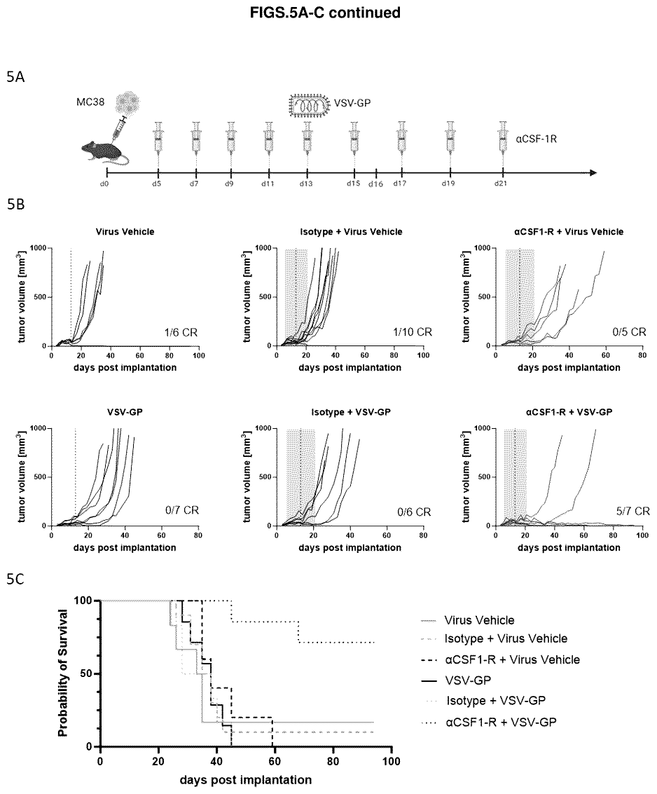

### FIGS. 5A-C

To study synergistic effects of macrophage depletion and VSV-GP based oncolytic virus therapy in an in vivo mouse tumor model, MC38 tumor-bearing mice were treated with α-CSF1-R and VSV-GP and tumor growth was monitored over time (FIG. 5A).

5/7 mice showed long-lasting and complete remissions up to 90 days following α-CSF1-R and VSV-GP treatment (FIGS. 5B&C). Although α-CSF1-R treatment without VSV-GP treatment resulted in a tumor growth delay compared to the isotype treated controls it did not show a long-lasting effect as being observed when α-CSF1-R and VSV-GP treatment was combined. VSV-GP treatment alone did not have substantial effects compared to the mock treated controls.

Thus, these data infer a synergistic effect of α-CSF1-R and VSV-GP, thereby pinpointing to a substantial role of macrophages depletion in facilitating OV spread.

### Example 4

### Synergistic Effects of αCSF-1R and VSV-GP in MC38 Tumors

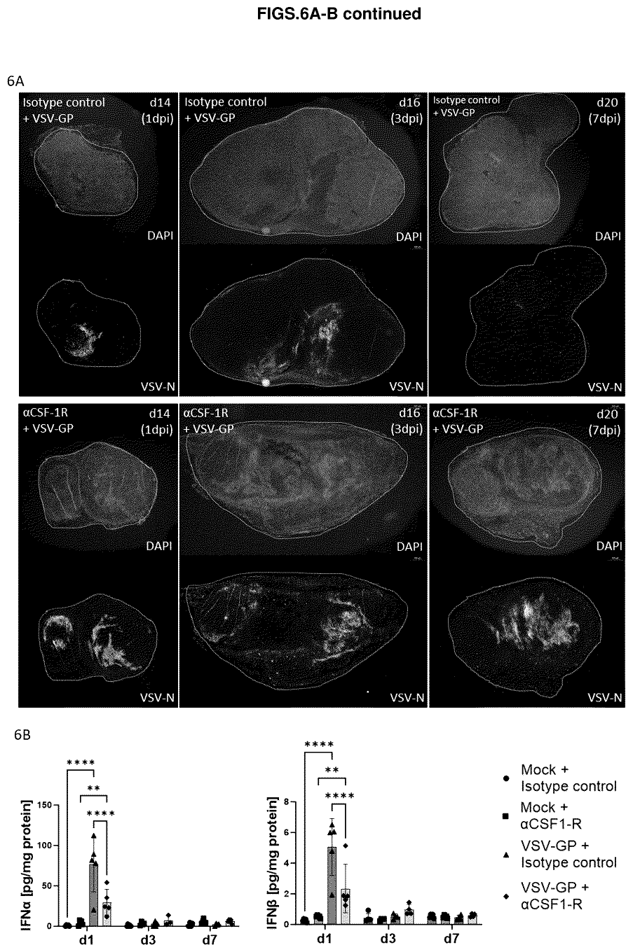

### FIGS. 6A-B

The earlier experiments were able to show that α-CSF1-R and VSV-GP could act synergistically in the MC38 murine tumor model. To learn about oncolytic virus spread and immediate immune responses, an in-depth multiplex analysis was performed at different time points after α-CSF1-R and VSV-GP treatment. MC38 cells were implanted s.c. into C57/Bl6 mice and the above validated treatment regimens of α-CSF1-R and VSV-GP were be applied. Tumor tissue was harvested at day 1, 3 and 7 post infection and subjected to IHC/IF and cytokine analyses (n=5 mice/time point). Immunofluorescence stainings of the N protein of VSV-GP revealed big positive clusters in α-CSF1-R and VSV-GP treated tumors indicative of viral replication in these cells at the respective harvesting timepoints (FIG. 6A). In comparison the isotype and VSV-GP treated tumors only showed some positive patches one day post infection and almost no VSV-N protein could be detected after 3 or 7 days post infection (dpi). Similarly, necrosis was increased in the combination therapy tumors compared to all other groups. These findings again indicate that depletion of macrophages is linked to an increased and prolonged viral replication and spread and therefore to a more efficient oncolysis. Finally, cytokine analysis of the tumors revealed significantly lower amounts of Interferon-α and β in the α-CSF1-R and VSV-GP combination group one day post viral treatment compared to the isotype and VSV-GP treated group (FIG. 6B) which again points to a better viral replication following α-CSF1-R depletion.

1. A method of treating cancer in a subject comprising administering an antagonistic antibody which is directed against CSF1 or CSFR to the subject and further comprising administering a recombinant vesicular stomatitis virus to the subject and thereby treating the cancer, wherein in said vesicular stomatitis virus the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV.

2. The method according to claim 1, wherein the cancer is a solid cancer.

3. The method according to claim 1, wherein the cancer is selected from the list consisting of: a reproductive tumor, an ovarian tumor, a testicular tumor, an endocrine tumor, a gastrointestinal tumor, a pancreatic tumor, a liver tumor, a kidney tumor, a colon tumor, a colorectal tumor, a bladder tumor, a prostate tumor, a skin tumor, melanoma, a respiratory tumor, a lung tumor, a breast tumor, a head & neck tumor, a head and neck squamous-cell carcinoma (HNSCC) and a bone tumor.

4. The method according to any one of the preceding claims, wherein the recombinant vesicular stomatitis virus is administered concomittantly, sequentially or alternately with the antagonistic antibody.

5. The method according to any one of claims 1-4, wherein the antagonistic antibody is selected from the group consisting of axatilimab, emactuzumab, cabiralizumab, AMG820LY3022855, or IMC-CS4.

6. A composition comprising an antagonistic antibody which is directed against CSF1 or CSFR and a recombinant vesicular stomatitis virus, wherein in said vesicular stomatitis virus the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV in said vesicular stomatitis virus.

7. A kit of parts comprising:
a) A recombinant vesicular stomatitis virus or a pharmaceutical composition comprising said recombinant vesicular stomatitis virus, wherein in said vesicular stomatitis virus the gene coding for the glycoprotein G of the vesicular stomatitis virus is replaced by the gene coding for the glycoprotein GP of lymphocyte choriomeningitis virus (LCMV), and/or the glycoprotein G is replaced by the glycoprotein GP of LCMV in said vesicular stomatitis virus, and
b) an antagonistic antibody which is directed against CSF1 or CSFR.
